SEQUENCE LISTING (29 sequences)

SEQ ID NO: 1 - protein, 330 aa, Agrocybe aegerita
  mat_peptide (1)..(330)
EPTQPPGPPEDTSAKLVNDKDHPWKPLRPGDIRGPCPGLNTLASHGYLPRNGVATPAQIINAVQEGFNMDNSVALFATYEAHLMVGNLLTDLLSIGRKTPLTGPDLPPPANIGGLSEHGLFEGDASMTRGDAFFGNNDEFNEELFQQFIDYSNRFGGGYYNLTVAVELRFKRIQDSIATNPEFNFVSPRFFAAYGESVAPNNFFVDGRKDDGHLDMDAARGFFQFGRMPDGFFRPNGTKGNAGLDDVVRAHPVQPGRNLGRVNSYTHDPTSADFTTPCLLYENFANKTVTALYPNPKGQLRRAIKANLHFLFLAINRTVGCAEVFPYGRD

SEQ ID NO: 2 - protein, 328 aa, Agrocybe aegerita
  mat_peptide (1)..(328)
EPGLPPGPLENSSAKLVNDEAHPWKPLRPGDIRGPCPGLNTLASHGYLPRNGVATPVQIINAVQEGLNFDNQAAVFATYAAHLVDGNLITDLLSIGRKTRLTGPDPPPPASVGGLNEHGTFEGDASMTRGDAFFGNNHDFNETLFEQLVDYSNRFGGGKYNLTVAGELRFKRIQDSIATNPNFSFVDFRFFTAYGETTFPANLFVDGRRDDGQLDMDAARSFFQFSRMPDDFFRAPSPRSGTGVEVVIQAHPMQPGRNVGKINSYTVDPTSSDFSTPCLMYEKFVNITVKSLYPNPTVHVRKALNTNLDFFFQGVAAGCTQVFPYGRD

SEQ ID NO: 3 - protein, 377 aa, Laccaria bicolor
MARLTFLAAIALALSSTTVLAFPSYGSLAGLSEAELDRIIPLLEARNAGPPPGPLKNTSTKLVNDKNHPWKPLGYGDIRGPCPGLNTLASHGWLPRNGIATPAQIVNAVQEGFNMGNDLAVFVTYAAHLVDGNQVTDLLSIGGKTPQTGPDPPAPAIVGGLNTHAVFEGDASMTRGDAFFGDNHSFNETQFDEFSAFSNKFGGGYYNLSVAAEFRWQRIQESIATNPNFSLISPRYFTAYAESVFPLVFFVDGRVSDGRLSLPNARGFFQNSQMPKDFFRPNQSIGLNEIGDGISAIASAHPIAPGKNEGVGNYVLDPTSADFDHFCLLYINFVNQTVKSLYPNPKGVLLDALKRNLNNFYGPLNGSDCEQIFPYGK

SEQ ID NO: 4 - protein, 344 aa, Coprinopsis cinerea
  mat_peptide (1)..(344)
TSKLPIVFPPPPPEPIKDPWLKLVNDRAHPWRPLRRGDVRGPCPGLNTLASHGYLPRDGVATPAQIITAVQEGFNMEYGIATFVTYAAHLVDGNPLTNLISIGGKTRKTGPDPPPPAIVGGLNTHAVFEGDASMTRGDFHLGDNFNFNQTLWEQFKDYSNRYGGGRYNLTAAAELRWARIQQSMATNGQFDFTSPRYFTAYAESVFPINFFTDGRLFTSNTTAPGPDMDSALSFFRDHRYPKDFHRAPVPSGARGLDVVAAAYPIQPGYNADGKVNNYVLDPTSADFTKFCLLYENFVLKTVKGLYPNPKGFLRKALETNLEYFYQSFPGSGGCPQVFPWGKSD

SEQ ID NO: 5 - protein, 351 aa, Coprinopsis cinerea
MVSCKLPLPLLTLAIALANVNAFPAYQSLGGLSKRQLETIIPGLPVVNPGPPPGPLADSTLKLVNDAAHPYQAPRPHLDHRGPCPGLNTLANHGYLPRSGIATPAQIVQAVMEGFNMENTFAKFVTYAAFLVDGNPITNLMSIGGKTWRTGIIEPPPPAIVGGLNTHAVFEGDTSMTRGDFHFGDNHSFNQTLFDQFVEYSNIHGGGFYNLTAATELRYQRIQQSIATNPEMSFVSPRWFTAILLQDEKFPDDFHRAPGPFSFEGLGYLVTRRPMPPGRNVGGVDNYVPDPNSADFNSFCKMYEDFVNDIVVALYPNPTGLLRRNLIKNLEYFWTGMFDPACTEVKPYGTL

SEQ ID NO: 6 - protein, 386 aa, Coprinopsis cinerea
MNGLFATVKLALVTLLASQSQFANAFPAWQSLGGLSERQLDEVMPMLKHRVPPPPPGPPAFTGAKLVNDKAHPFKPLKKGDVRGPCPGLNTLASHGYLPRNGVASPSQIIDAVQEGFNMENELARFTTYVAHLVDGNLVTDLLSIGEKTRKTGPDPPPPAIVGGLNNHGTFEGDASLTRGDAFFGDNHNFNQELFDQFKNFSAVYGNGFFNMTVAGELRFHRIQQSIATNPEFSLVGLRHLTAYAEASFPSLFFVDGRKTGAEAGQLDMATAESFFRDMMYPPDFFRPAAPVAGDAGAIFLAHPFQPGRNVGGVNNFTVDDSLGSLLDFCGFYENFVNKTLKALYPNPKGVLRRNLNINLQFFFESLPKDESGTPVCTQVFPYGRN

SEQ ID NO: 7 - protein, 341 aa, Coprinopsis cinerea
MLKPRVPPPPPGPLAFNGTKLVNDEDHPFMPPRKGDARGPCPGLNTLASHGYLPRNGIATPAQIINAVQEGFNMENEIARFTTYTAHLMDGNLVTDLLSIGPKTPKTGPDPPPPAIVGGLNNHGTFEGDASLSRADAFFGDNHSFDQELFDQFRNFSAIYGNGFFNMTVAAELRFHRIQQSIATNPEFSFAGLRHITAYAEASFPPIFFVDGRKTGAEAGQLDMAAAESFFKHMMYPPDFHRPAEPVNSDAQAVFEVHPFQPGRNVGGVNNYTVDESLGGLLDFCGFYENFVNKTIKGLYPNPTGVLKRNLNINLDFLFEALPKAGDGSQPCTQVFPYGHD

SEQ ID NO: 8 - protein, 325 aa, Coprinus radians
PPPEYVGPKLVNDADHPWEPLRPGDIRGPCPGLNTLASHGYLPRNGVATPAQIINAIVEGFNFNYEGAVFVTYFAHIVDGNLVTDLLSIGGKTNLTGEDTGAPAIIGGLNTHSVFEGDASMTRDDFHFGDNHSFNQTLFDQFVEYSNTYGGGFYNQEVAGHLRRRRIEQSIATNPEFDFTSPRFFTAFAESSFPYSFFVDGRITERPGGLSMENATLFFRDHKMPDDFWRAPEPTGGLNVLDIYRASGSPPAGRNVNGTNTFTPDPNSADFDNPCELYYDYVNRIVKSLYPNPTGILRDNLNIALGHVFDSMDFGDCEQLFPYGR

SEQ ID NO: 9 - protein, 14 aa, Artificial sequence
  Peroxygenase motif I
XXXXSXXXGXGXXN

SEQ ID NO: 10 - protein, 14 aa, Artificial sequence
  Peroxygenase motif II
GXGXXNXXXAXXXR

SEQ ID NO: 11 - protein, 12 aa, Artificial sequence
  Peroxygenase motif III
RXXRIXXSXATN

SEQ ID NO: 12 - protein, 10 aa, Artificial sequence
  Peroxygenase motif IV
SXATNXXXXX

SEQ ID NO: 13 - protein, 7 aa, Artificial sequence
  Peroxygenase motif V
PXXFXRX

SEQ ID NO: 14 - protein, 11 aa, Artificial sequence
  Peroxygenase motif VI
XXXXLYPNPXX

SEQ ID NO: 15 - protein, 10 aa, Artificial
  Peroxygenase motif VII
EXDXSXXRXD

SEQ ID NO: 16 - protein, 243 aa, Aspergillus niger
LATGSTKLLPWSPPGHGDVRGPCPMLNTLANHGLLPHNGKDISQEVITEVLNNTLNLADGLSAFLFEEAMTTVEDPKATTFSLSDLNCPGILEHDGSLSRQDTYFGNNHEFNQTIFDQTKSYWTTPLIDMYQAAEAHEARLNTSKATNPTFNLSETGLTFSFGETAAYMIVFEDTNLGYANRSWVEYFFENERLPQELGWTKRPFITTGQVLVDMTTWVINSTIGVTPEEQAEMQDFGKKITG

SEQ ID NO: 17 - protein, 259 aa, Aspergillus niger
LATGSTKLLPWSPPGHGDVRGPCPMLNTLANHGLLPHNGKDISQEVITEVLNNTLNLADGLSAFLFEEAMTTVEDPKATTFSLSDLNCPGILEHDGSLRYLPLQKHRGNQADLLSRQDTYFGNNHEFNQTIFDQTKSYWTTPLIDMYQAAEAHEARLNTSKATNPTFNLSETGLTFSFGETAAYMIVFEDTNLGYANRSWVEYFFENERLPQELGWTKRPFITTGQVLVDMTTWVINSTIGVTPEEQAEMQDFGKKITG

SEQ ID NO: 18 - protein, 247 aa, Aspergillus niger
FPQQGAPHPLPWSPPGPNDVRAPCPMLNTLANHGYLPHNGKDITERHTINALYNALGIEEELAIYLHQEAVTTNPAPNATTFSLNDLSRHDILEHDASLSRQDAYFGDNHDFNQTIFDETRSYWTSPIIDVKQAAVSRQARVNTSMATNPNYTMSELGDSFSYGETAAYIIVLGDKEKGLVNRSRVEYLFENERLPLDLGWSRAKENITFDDLSTMLQRIINATGGEMDFRATIALPRLVYIYYEEA

SEQ ID NO: 19 - protein, 247 aa, Aspergillus niger
FPQQGAPHPLPWSPPGPNDVRAPCPMLNTLANHGYLPHNGKDITERHTINALYNALGIEEELAIYLHQEAVTTNPAPNATTFSLNDLSRHDILEHDASLSRQDAYFGDNHDFNQTIFDETRSYWTSPIIDVKQAAVSRQARVNTSMATNPNYTMSELGDSFSYGETAAYIIVLGDKEKGLVNRSRVEYLFENERLPLDLGWSRAKENITFDDLSTMLQRIINATGGESEFDRELAKRGGVHVGSWRG

SEQ ID NO: 20 - protein, 259 aa, Poronia punctata
KAACPYGYGEFQPEQTSDARGPCPVLNTLANHGYLPRDGRHIDENRTLTALHDALNLDIDFGKFLFTAGRLSNPKANSTWFDLDHLSRHGIFEHDGSLSRQDHHFGEWSRFNQTVWNWTLEYLPDDMLDVQTVANARAQRMTRSNLTNPDFALSYLGYLFSVGEAAAVLSILGDKKTQTCPKAFADYIFVNERLPYELGWKKQDASISFDDLVETFEDLERHTSFPFPPPLDNSTDIFDQLVEGGQSKKKRCSAHIGCF

SEQ ID NO: 21 - protein, 259 aa, Chaetomium virescens
ELDFSKWKTRQPGEFRAPCPAMNSLANHGFIPRDGRNITVAMLVPVLQEVFHLSPELAQTISTLGLFTAQDPSKGVFTLDDLNRHNLFEHDASLSREDYYFHKDASTFRPEVFKKFMSHFKGKEYVTLEDAASARYAMVQESRKKNPTFTYTVQQRITSYGETIKYFRTIVEPATGKCPVAWIKILFEQERLPYNEGWRPPKAELSGFSMASDVLELALVTPEKLIDKPCEGKQCPQARGIHGYFGMLLPITAQELAVK

SEQ ID NO: 22 - protein, 247 aa, Chaetomium virescens
ELDFSKWKTRQPGEFRAPCPAMNSLANHGFIPRDGRNITVAMLVPVLQEVFHLSPELAQTISTLGLFTAQDPSKGVFTLDDLNRHNLFEHDASLSREDYYFHKDASTFRPEVFKKFMSHFKGKEYVTLEDAASARYAMVQESRKKNPTFTYTVQQRITSYGETIKYFRTIVEPATGKCPVAWIKILFEQERLPYNEGWRPPKAELSGFSMASDVLELALVTPEKLIDKPCEGKQCPQARGIHGYFGM

SEQ ID NO: 23 - protein, 261 aa, Humicola insolens
GFHDWEPPGPNDVRAPCPMLNTLANHGFLPHHGRDLTRKQVVDGLYNGLNINKTAASTLFDFALMTSPKPNATTFSLDDLGRHNILEHDASLSRTDAYFGDVLAFNKTIFEETKRHWGKSPILDVTAAARARLGRIQTSKATNPEYFMSELGNIFTYGESVAYIMLIGDAKTGRANRRWVEYWFENERLPTHLGWRRPSKELTSDVLDTYISLIQNITLTLPGGTDPVKRRAASHFVFPFGQGLGGPAGVALMLISVVAYG

SEQ ID NO: 24 - protein, 242 aa, Chaetomium globosum
GFDTWAPPGPYDVRGPCPMLNTLTNHGFFPHDGQDIDRETTENALFDALHVNKTLASFLFDFALTTNPIANSTTFSLNDLGNHNVLEHDASLSRADAYHGSVLAFNHTIFEETKSYWTDETVTLKMAADARYYRIKSSQATNPTYQMSELGDAFTYGESAAYVVLFGDKESQTVPRSWVEWLFEKEQLPQHLGWKRPATSFELNDLDKFMALIQNYTQEIEEPSCESRKQRRKPRGPSHFGF

SEQ ID NO: 25 - protein, 301 aa, Chaetomium globosum
GFDTWAPPGPYDVRAPCPMLNTLANHGFLPHDGHEITREQTENALFDALHIDKMLGSSLFDFAMTTNPVANSTTFSLNDLGNHNVLEHDASLSRSDAYFGNTLTFNQTVFDETKSYWTDETVTIEMASNARLARIKTSNATNPTYSMSELGNGFTKGESAAYVVIFGDKISGTVPRAWVEWLFEHEQLPQHLGWKRPTELFRDGDLDKYMDAMQNVIVEIALKTQPSTPSIKPTQTPSSPTRLLLKRLGRQLMLIVPRPIRLRVLRNTPPLRLITKNKPREMAPNLLILAVHKRATSMQKR

SEQ ID NO: 26 - protein, 344 aa, Chaetomium globosum
GFDTWAPPGPYDVRAPCPMLNTLANHGFLPHDGHEITREQTENALFDALHIDKMLGSSLFDFAMTTNPVANSTTFSLNDLGNHNVLEHDASLSRSDAYFGNTLTFNQTVFDETKSYWTDETVTIEMASNARLARIKTSNATNPTYSMSELGNGFTKGESAAYVVIFGDKISGTVPRAWVEWLFEHEQLPQHLGWKRPTELFRDGDLDKYMDAMQNVIVGETPGCPAGKQQQRKGRRTPSHFGWDPTPVNKLASLRIAGYCHEIALKTQPSTPSIKPTQTPSSPTRLLLKRLGRQLMLIVPRPIRLRVLRNTPPLRLITKNKPREMAPNLLILAVHKRATSMQKR

SEQ ID NO: 27 - protein, 243 aa, Chaetomium globosum
GFDTWAPPGPYDVRAPCPMLNTLANHGFLPHDGHEITREQTENALFDALHIDKMLGSSLFDFAMTTNPVANSTTFSLNDLGNHNVLEHDASLSRSDAYFGNTLTFNQTVFDETKSYWTDETVTIEMASNARLARIKTSNATNPTYSMSELGNGFTKGESAAYVVIFGDKISGTVPRAWVEWLFEHEQLPQHLGWKRPTELFRDGDLDKYMDAMQNVIVGETPGCPAGKQQQRKGRRTPSHFGW

SEQ ID NO: 28 - protein, 276 aa, Sclerotinia sclerotiorum
MKLNFLSTTLALGLVSARAHYQQQIIANDTEGEWIAPSATDYRGPCPMLNTLANHGFLPRDGRNLTEHNVVKGLNHGLNFNKSLGSIMFQHAVPASPAYPNTTFFTLDDLNRHNVLEHDASISRSDAYFGNNHIFNQTIFDTTKMYWPSETLTAQHLIDGKIFRQIVSRTTNPNYTFTSTTQAFSLGEMAAPIVAFGDKNALTANRTLVESWIENERLPTELGWSKPEEEVSLGDILYVTGALANLTSLLSDVVITPRGESAGAHAKRMGHWGVSM

SEQ ID NO: 29 - protein, 259 aa, Aspergillus carbonarius
MKSTILLITTSLSQALAQVSSHPFPWSAPGPNDVRGPCPMLNTLANHGFLPHDGKDITEDRIVMVLNNSLNLDEELSQFLFKEALTTNPDPNATTFSLNDLSRHNILEHDASLSRQDYYFGDNHDFNQTVFNETRSYWTAPLIDFNAAAQARLARVNTSMATNPTYTESETGLAFSYGESAAYMIVFAEGSETANRSWVEYFFEHERLPQQLGWTKPQESISSSVLIDTVTGIANASNASSLVVAELLDFVALHMGRLP

(19) United States
(12) Patent Application Publication
(10) Pub. No.: US 2014/0234917 A1
(43) Pub. Date: Aug. 21, 2014

(54) Enzymatic Preparation of Diols

(71) Applicants: Henrik Lund, Vaerloese (DK); Jesper Brask, Vaerloese (DK); Lisbeth Kalum, Vaerloese (DK); Ana Gutierrez Suarez, Sevilla (ES); Esteban Daniel Babot, Sevilla (ES); Rene Ullrich, Zittau (DE); Martin Hofrichter, Dresden (DE); Angel Tomas Martinez Ferrer, Madrid (ES); Jose Carlos Del Rio Andrade, Sevilla (ES)

(72) Inventors: Henrik Lund, Vaerloese (DK); Jesper Brask, Vaerloese (DK); Lisbeth Kalum, Vaerloese (DK); Ana Gutierrez Suarez, Sevilla (ES); Esteban Daniel Babot, Sevilla (ES); Rene Ullrich, Zittau (DE); Martin Hofrichter, Dresden (DE); Angel Tomas Martinez Ferrer, Madrid (ES); Jose Carlos Del Rio Andrade, Sevilla (ES)

(73) Assignee: Novozymes A/S, Bagsvaerd (DK)

(21) Appl. No.: 14/131,165

(22) PCT Filed: Jun. 29, 2012
(86) PCT No.: PCT/EP2012/062763

§ 371 Date: Apr. 4, 2014

Related U.S. Application Data
(60) Provisional application No. 61/506,553, filed on Jul. 11, 2011.

(30) Foreign Application Priority Data
Jul. 7, 2011 (EP) 11173073.5

(51) Int. Cl. C12P 7/04; C12P 7/64; C12P 7/18

(52) U.S. Cl. CPC C12P 7/04; C12P 7/18; C12P 7/6409; C12P 7/6427
USPC 435/129; 435/157; 435/148; 435/136; 435/158; 435/146; 435/134

(57) ABSTRACT

The invention relates to enzymatic methods for hydroxylation in position 2 or 3 of two ends of a substituted or unsubstituted, linear or branched aliphatic hydrocarbons.

### REFERENCE TO A SEQUENCE LISTING

This application contains a Sequence Listing in computer readable form. The computer readable form is incorporated herein by reference.

### BACKGROUND OF THE INVENTION

1. Field of the Invention

The present invention relates to the use of polypeptides having peroxygenase activity for site specific oxidation of aliphatic hydrocarbons.

2. Background

A peroxygenase denoted AaP from the agaric basidiomycete strain Agrocybe aegerita (strain TM-A1) was found to oxidize aryl alcohols and aldehydes. The AaP peroxygenase was purified from A. aegerita TM A1 by several steps of ion chromatography and SDS-PAGE, the molecular weight was determined and the N-terminal 14 amino acid sequence was determined after 2-D electrophoresis but the encoding gene was not isolated (Ullrich et al., 2004, Appl. Env. Microbiol. 70(8): 4575-4581).

WO 2006/034702 discloses methods for the enzymatic hydroxylation of non-activated hydrocarbons, such as, naphtalene, toluol and cyclohexane, using the AaP peroxygenase enzyme of Agrocybe aegerita TM A1. This is also described in Ullrich and Hofrichter, 2005, FEBS Letters 579: 6247-6250.

WO 2008/119780 discloses eight different peroxygenases from Agrocybe aegerita, Coprinopsis cinerea, Laccaria bicolor and Coprinus radians; also shown as SEQ ID NOs:1-8 in the present application.

DE 103 32 065 A1 discloses methods for the enzymatic preparation of acids from alcohols through the intermediary formation of aldehydes by using the AaP peroxygenase enzyme of Agrocybe aegerita TM A1.

A method was reported for the rapid and selective spectrophotometric direct detection of aromatic hydroxylation by the AaP peroxygenase (Kluge et al., 2007, Appl Microbiol Biotechnol 75: 1473-1478).

It is well-known that a direct regioselective introduction of oxygen functions (oxygenation) into organic molecules constitutes a problem in chemical synthesis. It is particularly difficult to catalyse the selective hydroxylation of aliphatic hydrocarbons. The products may be used as important intermediates in a wide variety of different syntheses.

In particular the chemical hydroxylation of alkanes is relatively complex, requires aggressive/toxic chemicals/catalysts and leads to a series of undesired by-products.

It is known that an intracellular enzyme, methane monooxygenase (MMO, EC 14.13.25), oxygenates/hydroxylates the terminal carbon of some hydrocarbons. The MMO enzyme consists of several protein components and is formed by methylotrophic bacteria (e.g., Methylococcus capsulatus); it requires complex electron donors such as NADH or NADPH, auxiliary proteins (flavin reductases, regulator protein) and molecular oxygen (O₂). The natural substrate of MMO is methane, which is oxidized to methanol. As a particularly unspecific biocatalyst, MMO oxygenates/hydroxylates, as well as methane, a series of further substrates such as n-alkanes and their derivatives, cycloalkanes, aromatics, carbon monoxide and heterocycles. Utilization of the enzyme in biotechnology is currently not possible, since it is difficult to isolate, like most intracellular enzymes, it is of low stability, and the cosubstrates required are relatively expensive.

### SUMMARY OF THE INVENTION

In a first aspect, the inventors of the present invention have provided an enzymatic method for introducing a hydroxy or an oxo group, at the second or third carbon of at least two ends of a substituted or unsubstituted, linear or branched, aliphatic hydrocarbon having at least five carbons and having a hydrogen attached to said second or third carbon, comprising contacting the aliphatic hydrocarbon with hydrogen peroxide and a polypeptide having peroxygenase activity; wherein the polypeptide comprises:

a) an amino acid sequence which has at least 30% identity to SEQ ID NO: 1, 2, 3, 4, 5, 6, 7, 8, 16, 17, 18, 19, 20, 21, 22, 23, 24, 25, 26, 27, 28 or 29; and
b) an amino acid sequence represented by one or more of the following motifs:

(SEQ ID NO: 9)

| | |
|---|---|
| Motif I: | [FL]XX[YF]S[AN]X[FHY]G[GN]GX[YF]N; |

(SEQ ID NO: 10)

| | |
|---|---|
| Motif II: | G[GN]GX[YF]NXX[VA]AX[EH][LF]R; |

(SEQ ID NO: 11)

| | |
|---|---|
| Motif III: | RXXRI[QE][DEQ]S[IM]ATN; |

(SEQ ID NO: 12)

| | |
|---|---|
| Motif IV: | S[IM]ATN[PG][EQN][FM][SDN][FL]; |

(SEQ ID NO: 13)

| | |
|---|---|
| Motif V: | P[PDK][DG]F[HFW]R[AP]; |

(SEQ ID NO: 14)

| | |
|---|---|
| Motif VI: | [TI]XXXLYPNP[TK][GV]; |

(SEQ ID NO: 15)

| | |
|---|---|
| Motif VII: | E[HG]DXSX[ST]RXD. |

In further aspects, the invention provides uses of polypeptides having peroxygenase activity for removal of lipid containing stains from laundry; and for reducing unpleasant odor from laundry.

### BRIEF DESCRIPTION OF THE FIGURES

FIG. 1 shows a chromatographic profile of tetradecane incubated with C. cinerea peroxygenase (0.5 mM H₂O₂); from Example 3.

FIG. 2 shows a chromatographic profile of tetradecane incubated with A. aegerita peroxygenase (2.5 mM H₂O₂); from Example 3.

FIG. 3 shows a chromatographic profile of tetradecanol incubated with C. cinerea peroxygenase (0.5 mM H₂O₂); from Example 4.

FIG. 4 shows a chromatographic profile of tetradecanol incubated with A. aegerita peroxygenase (2.5 mM H₂O₂); from Example 4.

### DEFINITIONS

Peroxygenase Activity:

The term “peroxygenase activity” is defined herein as “unspecific peroxygenase” according to EC 1.11.2.1. This is a heme-thiolate protein. Enzymes of this type include glycoproteins secreted by agaric basidiomycetes. They catalyse the insertion of an oxygen atom from H₂O₂ into a wide variety of substrates, such as naphthalene, 4-nitrobenzodioxole; and alkanes such as propane, hexane and cyclohexane. They have little or no activity toward chloride.

For purposes of the present invention, peroxygenase activity is determined according to the spectrophotometric procedure described by Kluge et al. (2007, Appl Microbiol Biotechnol 75: 1473-1478).

Isolated Polypeptide:

The term “isolated polypeptide” as used herein refers to a polypeptide that is isolated from a source. In a preferred aspect, the polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE.

Substantially Pure Polypeptide:

The term “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptides of the present invention are preferably in a substantially pure form, i.e., that the polypeptide preparation is essentially free of other polypeptide material with which it is natively or recombinantly associated. This can be accomplished, for example, by preparing the polypeptide by well-known recombinant methods or by classical purification methods.

Mature Polypeptide:

The term “mature polypeptide” is defined herein as a polypeptide having peroxygenase activity that is in its final form following translation and any post-translational modifications, such as N-terminal processing, C-terminal truncation, glycosylation, phosphorylation, etc. In a preferred aspect, the mature polypeptide has the amino acid sequence shown in positions 1 to 330 of SEQ ID NO:1 based on the N-terminal peptide sequencing data (Ullrich et al., 2004, Appl. Env. Microbiol. 70(8): 4575-4581), elucidating the start of the mature protein of AaP peroxygenase enzyme. In another preferred aspect, the mature polypeptide has the amino acid sequence shown in positions 1 to 328 of SEQ ID NO:2.

Identity:

The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.orq), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; http://emboss.orq), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

Modification:

The term “modification” means herein any chemical modification of the polypeptide consisting of the mature polypeptide of SEQ ID NO: 1, 2, 3, 4, 5, 6, 7, 8, 16, 17, 18, 19, 20, 21, 22, 23, 24, 25, 26, 27, 28 or 29; or a homologous sequence thereof; as well as genetic manipulation of the DNA encoding such a polypeptide. The modification can be a substitution, a deletion and/or an insertion of one or more (several) amino acids as well as replacements of one or more (several) amino acid side chains.

### DETAILED DESCRIPTION OF THE INVENTION

### Polypeptides Having Peroxygenase Activity (Peroxygenases)

The present invention relates to uses of an isolated polypeptide, which is preferably recombinantly produced, having peroxygenase activity, which comprises an amino acid sequence having at least 30% identity, preferably at least 35%, 40%, 45%, 50%, 55%, 60%, 65%, 70%, 75%, 80%, 85%, 90%, 95%, 97%, or 98% identity to the polypeptide of SEQ ID NO: 1, 2, 3, 4, 5, 6, 7, 8, 16, 17, 18, 19, 20, 21, 22, 23, 24, 25, 26, 27, 28 or 29; preferably SEQ ID NO:2 or SEQ ID NO:4.

In a preferred embodiment, the polypeptide comprises an amino acid sequence represented by one or more of the following motifs, preferably comprising two or more, three or more, four or more, five or six of the following motifs:

(SEQ ID NO: 9)

| | |
|---|---|
| Motif I: | [FL]XX[YF]S[AN]X[FHY]G[GN]GX[YF]N; |

(SEQ ID NO: 10)

| | |
|---|---|
| Motif II: | G[GN]GX[YF]NXX[VA]AX[EH][LF]R; |

(SEQ ID NO: 11)

| | |
|---|---|
| Motif III: | RXXRI[QE][DEQ]S[IM]ATN; |

(SEQ ID NO: 12)

| | |
|---|---|
| Motif IV: | S[IM]ATN[PG][EQN][FM][SDN][FL]; |

(SEQ ID NO: 13)

| | |
|---|---|
| Motif V: | P[PDK][DG]F[HFW]R[AP]; |

(SEQ ID NO: 14)

| | |
|---|---|
| Motif VI: | [TI]XXXLYPNP[TK][GV]; |

(SEQ ID NO: 15)

| | |
|---|---|
| Motif VII: | E[HG]DXSX[ST]RXD. |

In a more preferred embodiment, the peroxygenase comprises an amino acid sequence represented by the motif: E[HG]DXSX[ST]RXD.

In another embodiment, the polypeptide comprises an amino acid sequence having a substitution, deletion, and/or insertion of one or several amino acids of the mature polypeptide of SEQ ID NO: 1, 2, 3, 4, 5, 6, 7, 8, 16, 17, 18, 19, 20, 21, 22, 23, 24, 25, 26, 27, 28 or 29; preferably SEQ ID NO:2 or SEQ ID NO:4.

In yet another embodiment, the polypeptide of the first aspect comprises or consists of the amino acid sequence of SEQ ID NO: 1, 2, 3, 4, 5, 6, 7, 8, 16, 17, 18, 19, 20, 21, 22, 23, 24, 25, 26, 27, 28 or 29; preferably SEQ ID NO:2 or SEQ ID NO:4; or a fragment thereof having peroxygenase activity; preferably the polypeptide comprises or consists of the mature polypeptide of SEQ ID NO: 1, 2, 3, 4, 5, 6, 7, 8, 16, 17, 18, 19, 20, 21, 22, 23, 24, 25, 26, 27, 28 or 29; preferably SEQ ID NO:2 or SEQ ID NO:4.

Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., peroxygenase activity) to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to a polypeptide according to the invention.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

The total number of amino acid substitutions, deletions and/or insertions of the mature polypeptide of SEQ ID NO: 1, 2, 3, 4, 5, 6, 7, 8, 16, 17, 18, 19, 20, 21, 22, 23, 24, 25, 26, 27, 28 or 29; preferably SEQ ID NO:2 or SEQ ID NO:4; is 10, preferably 9, more preferably 8, more preferably 7, more preferably at most 6, more preferably 5, more preferably 4, even more preferably 3, most preferably 2, and even most preferably 1.

Another preferred embodiment relates to the polypeptide having peroxygenase activity of the first aspect of the invention, wherein the mature polypeptide is amino acids 1 to 330 of SEQ

ID NO:1.

Yet another preferred embodiment relates to the polypeptide having peroxygenase activity of the first aspect of the invention, wherein the mature polypeptide is amino acids 1 to 328 of SEQ ID NO:2.

Yet another preferred embodiment relates to the polypeptide having peroxygenase activity of the first aspect of the invention, wherein the mature polypeptide is amino acids 1 to 344 of SEQ ID NO:4.

Yet another preferred embodiment relates to the polypeptide having peroxygenase activity of the first aspect of the invention, wherein the mature polypeptide is amino acids 1 to 261 of SEQ ID NO:23.

### Hydrogen Peroxide

The hydrogen peroxide required by the peroxygenase may be provided as an aqueous solution of hydrogen peroxide or a hydrogen peroxide precursor for in situ production of hydrogen peroxide. Any solid entity which liberates upon dissolution a peroxide which is useable by peroxygenase can serve as a source of hydrogen peroxide. Compounds which yield hydrogen peroxide upon dissolution in water or an appropriate aqueous based medium include but are not limited to metal peroxides, percarbonates, persulphates, perphosphates, peroxyacids, alkyperoxides, acyl peroxides, peroxyesters, urea peroxide, perborates and peroxycarboxylic acids or salts thereof.

Another source of hydrogen peroxide is a hydrogen peroxide generating enzyme system, such as an oxidase together with a substrate for the oxidase. Examples of combinations of oxidase and substrate comprise, but are not limited to, amino acid oxidase (see e.g., U.S. Pat. No. 6,248,575) and a suitable amino acid, glucose oxidase (see e.g., WO 95/29996) and glucose, lactate oxidase and lactate, galactose oxidase (see e.g., WO 00/50606) and galactose, and aldose oxidase (see e.g., WO 99/31990) and a suitable aldose.

By studying EC 1.1.3._, EC 1.2.3._, EC 1.4.3._, and EC 1.5.3._or similar classes (under the International Union of Biochemistry), other examples of such combinations of oxidases and substrates are easily recognized by one skilled in the art.

Hydrogen peroxide or a source of hydrogen peroxide may be added at the beginning of or during the method of the invention, e.g., as one or more separate additions of hydrogen peroxide; or continously as fed-batch addition. Typical amounts of hydrogen peroxide correspond to levels of from 0.001 mM to 25 mM, preferably to levels of from 0.005 mM to 5 mM, and particularly to levels of from 0.01 to 1 mM hydrogen peroxide. Hydrogen peroxide may also be used in an amount corresponding to levels of from 0.1 mM to 25 mM, preferably to levels of from 0.5 mM to 15 mM, more preferably to levels of from 1 mM to 10 mM, and most preferably to levels of from 2 mM to 8 mM hydrogen peroxide.

### Surfactants

The method of the invention may include application of a surfactant (for example, as part of a detergent formulation or as a wetting agent). Surfactants suitable for being applied may be non-ionic (including semi-polar), anionic, cationic and/or zwitterionic; preferably the surfactant is anionic (such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap) or non-ionic (such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”)), or a mixture thereof.

When included in the method of the invention, the concentration of the surfactant will usually be from about 0.01% to about 10%, preferably about 0.05% to about 5%, and more preferably about 0.1% to about 1% by weight.

### Aliphatic Hydrocarbons

The hydrocarbons, which are oxidized in the method of the invention, are aliphatic hydrocarbons having a chain of at least five carbons. Preferably, the aliphatic hydrocarbon is an alkane or an alkene; more preferably, the aliphatic hydrocarbon is an alkane, such as pentane, hexane, heptane, octane, nonane, decane, undecane, dodecane, tridecane, tetradecane, pentadecane or hexadecane, or isomers thereof. Even more preferably, the aliphatic hydrocarbon is undecane, dodecane, tridecane, tetradecane, pentadecane or hexadecane, or isomers thereof.

In an embodiment, the aliphatic hydrocarbon is not n-hexane or n-decane.

The aliphatic hydrocarbons are linear or branched, but not cyclic, as site specific oxidation is not possible with cyclic hydrocarbons. Branched hydrocarbons correspond to isomers of linear hydrocarbons.

The aliphatic hydrocarbons are substituted or unsubstituted. Preferably, the aliphatic hydrocarbons are unsubstituted, such as non-activated hydrocarbons.

When the aliphatic hydrocarbons are substituted (functional groups attached), the preferred substituents are halogen, hydroxyl, carboxyl, amino, nitro, cyano, thiol, sulphonyl, formyl, acetyl, methoxy, ethoxy, phenyl, benzyl, xylyl, carbamoyl and sulfamoyl; more preferred substituents are chloro, hydroxyl, carboxyl and sulphonyl; and most preferred substituents are chloro and carboxyl.

The aliphatic hydrocarbons may be substituted by up to 10 substituents, up to 8 substituents, up to 6 substituents, up to 4 substituents, up to 2 substituents, or by up to one substituent.

### Methods and Uses

The present invention provides a method for site specific introduction of a hydroxy and/or an oxo (keto) group at the second or third carbon of at least two ends of an aliphatic hydrocarbon, using a peroxygenase and hydrogen peroxide.

The aliphatic hydrocarbon must include a chain of at least five carbons. The second and third carbons are determined by counting the carbon atoms from any end of the aliphatic hydrocarbon.

The aliphatic hydrocarbon must have at least one hydrogen attached to a carbon which is hydroxylated by attachment of a hydroxy group; and at least two hydrogens attached to a carbon when an oxo group is introduced. In a preferred embodiment, the second or third carbon is unsubstituted before being contacted with the peroxygenase.

According to the method of the invention, the hydroxy and/or oxo groups are introduced independently of each other at the (at least) two ends of the aliphatic hydrocarbon. Thus, a hydroxy group can be introduced at one end, at the same time as an oxo group is introduced at another (the other) end—and vice versa. Two hydroxy groups, or two oxo groups, or one hydroxy group and one oxo group, cannot be introduced at the same end of the aliphatic hydrocarbon. Some examples of combinations are shown in Example 1.

In the context of the present invention, “oxidation” means introduction of a hydroxy and/or an oxo group.

Accordingly, in a first aspect, the present invention provides a method for introducing a hydroxy and/or an oxo (keto) group at the second or third carbon of (at least) two ends of a substituted or unsubstituted, linear or branched, aliphatic hydrocarbon having at least five carbons and having at least one hydrogen attached to said second or third carbon, comprising contacting the aliphatic hydrocarbon with hydrogen peroxide and a polypeptide having peroxygenase activity; wherein the polypeptide comprises:

a) an amino acid sequence which has at least 30% identity to SEQ ID NO: 1, 2, 3, 4, 5, 6, 7, 8, 16, 17, 18, 19, 20, 21, 22, 23, 24, 25, 26, 27, 28 or 29; preferably SEQ ID NO:2 or SEQ ID NO:4; and
b) an amino acid sequence represented by one or more of the following motifs:

(SEQ ID NO: 9)

| | |
|---|---|
| Motif I: | [FL]XX[YF]S[AN]X[FHY]G[GN]GX[YF]N; |

(SEQ ID NO: 10)

| | |
|---|---|
| Motif II: | G[GN]GX[YF]NXX[VA]AX[EH][LF]R; |

(SEQ ID NO: 11)

| | |
|---|---|
| Motif III: | RXXRI[QE][DEQ]S[IM]ATN; |

(SEQ ID NO: 12)

| | |
|---|---|
| Motif IV: | S[IM]ATN[PG][EQN][FM][SDN][FL]; |

(SEQ ID NO: 13)

| | |
|---|---|
| Motif V: | P[PDK][DG]F[HFW]R[AP]; |

(SEQ ID NO: 14)

| | |
|---|---|
| Motif VI: | [TI]XXXLYPNP[TK][GV]; |

(SEQ ID NO: 15)

| | |
|---|---|
| Motif VII: | E[HG]DXSX[ST]RXD; |
| | preferably, |
| Motif VII: | E[HG]DXSX[ST]RXD. |

In an embodiment, the aliphatic hydrocarbon is not n-hexane or n-decane.

In a preferred embodiment, the aliphatic hydrocarbon is oxidized to (converted to) a diol, by introduction of two hydroxy groups. More preferably, the two hydroxy groups are located at each end of a linear aliphatic hydrocarbon.

The method of the invention may be used for a variety of purposes, like bulk chemical synthesis (biocatalysis), increasing aqueous solubility of aliphatic hydrocarbons, bioremediation, and modification of the characteristics of food products.

The method of the invention may also be used for a number of industrial processes in which said oxidation reactions are beneficial. An example of such use is in the manufacture of pulp and paper products where alkanes and other relevant aliphatic hydrocarbons that are present in the wood (resin) can result in depositioning problems in the pulp and paper manufacturing process. These hydrophobic compounds are the precursors of the so-called pitch deposits within the pulp and paper manufacturing processes. Pitch deposition results in low quality pulp, and can cause the shutdown of pulp mill operations. Specific issues related to pulps with high extractives content include runnability problems, spots and holes in the paper, and sheet breaks. Treatment with peroxygenase can increase the solubility of said compounds and thereby mitigate problems.

Yet another use of the method of the invention is in, for example, oil or coal refineries where the peroxygenase catalyzed oxidation can be used to modify the solubility, viscosity and/or combustion characteristics of hydrocarbons. Specifically the treatment can lead to changes in the smoke point, the kindling point, the fire point and the boiling point of the hydrocarbons subjected to the treatment.

In the synthesis of bulk chemicals, agro chemicals (incl. pesticides), specialty chemicals and pharmaceuticals the method of the invention may obviously be relevant in terms of selectively introducing hydroxy groups in the substrates thereby affecting the solubility of the modified compound. Furthermore, the selective oxidation provides a site for further modification by methods known in the art of organic chemical synthesis and chemo-enzymatic synthesis.

Natural gas is extensively processed to remove higher alkanes. Oxidation of such higher alkanes may be used to improve water solubility, and thus facilitate removal of the higher alkanes by washing the natural gas stream. Removal may be performed at the well or during refining.

Oxidation, according to the invention, of oil waste will significantly improve biodegradability and will be applicable both in connection with waste water treatment from refineries and bioremediation of contaminated ground or water

The methods of the invention may be carried out with an immobilized polypeptide having peroxygenase activity (peroxygenase).

The methods of the invention may be carried out in an aqueous solvent (reaction medium), various alcohols, ethers, other polar or non-polar solvents, or mixtures thereof. By studying the characteristics of the aliphatic hydrocarbon used in the methods of the invention, suitable examples of solvents are easily recognized by one skilled in the art. By raising or lowering the pressure at which the oxidation is carried out, the solvent (reaction medium) and the aliphatic hydrocarbon can be maintained in a liquid phase at the reaction temperature.

The methods according to the invention may be carried out at a temperature between 0 and 90 degrees Celsius, preferably between 5 and 80 degrees Celsius, more preferably between 10 and 70 degrees Celsius, even more preferably between 15 and 60 degrees Celsius, most preferably between 20 and 50 degrees Celsius, and in particular between 20 and 40 degrees Celsius.

The methods of the invention may employ a treatment time of from 10 seconds to (at least) 24 hours, preferably from 1 minute to (at least) 12 hours, more preferably from 5 minutes to (at least) 6 hours, most preferably from 5 minutes to (at least) 3 hours, and in particular from 5 minutes to (at least) 1 hour.

Diols (di-hydroxy aliphatic hydrocarbons) produced by the method of the invention may be used for producing polyurethan. Polyurethane is a polymer composed of a chain of organic units joined by carbamate (urethane) links. Polyurethane polymers are formed through step-growth polymerization, by reacting a monomer (with at least two isocyanate functional groups) with another monomer (with at least two hydroxyl groups) in the presence of a catalyst.

In another aspect, the present invention provides a method for introducing an oxo (keto) group at the second or third carbon of a substituted or unsubstituted, linear or branched, aliphatic hydrocarbon having at least five carbons and having at least two hydrogens attached to said second or third carbon, comprising contacting the aliphatic hydrocarbon with hydrogen peroxide and a polypeptide having peroxygenase activity; wherein the polypeptide comprises:

a) an amino acid sequence which has at least 30% identity to SEQ ID NO: 1, 2, 3, 4, 5, 6, 7, 8, 16, 17, 18, 19, 20, 21, 22, 23, 24, 25, 26, 27, 28 or 29; preferably SEQ ID NO:2 or SEQ ID NO:4; and
b) an amino acid sequence represented by one or more of the following motifs:

(SEQ ID NO: 9)

| | |
|---|---|
| Motif I: | [FL]XX[YF]S[AN]X[FHY]G[GN]GX[YF]N; |

(SEQ ID NO: 10)

| | |
|---|---|
| Motif II: | G[GN]GX[YF]NXX[VA]AX[EH][LF]R; |

(SEQ ID NO: 11)

| | |
|---|---|
| Motif III: | RXXRI[QE][DEQ]S[IM]ATN; |

(SEQ ID NO: 12)

| | |
|---|---|
| Motif IV: | S[IM]ATN[PG][EQN][FM][SDN][FL]; |

(SEQ ID NO: 13)

| | |
|---|---|
| Motif V: | P[PDK][DG]F[HFW]R[AP]; |

(SEQ ID NO: 14)

| | |
|---|---|
| Motif VI: | [TI]XXXLYPNP[TK][GV]; |

(SEQ ID NO: 15)

| | |
|---|---|
| Motif VII: | E[HG]DXSX[ST]RXD; |
| | preferably, |
| Motif VII: | E[HG]DXSX[ST]RXD. |

In an embodiment, the aliphatic hydrocarbon is not n-hexane or n-decane.

In yet another aspect, the present invention also provides a method for introducing a hydroxy or an oxo group at a terminal carbon of a linear or branched aliphatic hydrocarbon having at least five carbons, which is substituted with a carboxy group, comprising contacting the aliphatic hydrocarbon with hydrogen peroxide and a polypeptide having peroxygenase activity; wherein the polypeptide comprises:

a) an amino acid sequence which has at least 30% identity to SEQ ID NO: 1, 2, 3, 4, 5, 6, 7, 8, 16, 17, 18, 19, 20, 21, 22, 23, 24, 25, 26, 27, 28 or 29; preferably SEQ ID NO:2 or SEQ ID NO:4; and
b) an amino acid sequence represented by one or more of the following motifs:

(SEQ ID NO: 9)

| | |
|---|---|
| Motif I: | [FL]XX[YF]S[AN]X[FHY]G[GN]GX[YF]N; |

(SEQ ID NO: 10)

| | |
|---|---|
| Motif II: | G[GN]GX[YF]NXX[VA]AX[EH][LF]R; |

(SEQ ID NO: 11)

| | |
|---|---|
| Motif III: | RXXRI[QE][DEQ]S[IM]ATN; |

(SEQ ID NO: 12)

| | |
|---|---|
| Motif IV: | S[IM]ATN[PG][EQN][FM][SDN][FL]; |

(SEQ ID NO: 13)

| | |
|---|---|
| Motif V: | P[PDK][DG]F[HFW]R[AP]; |

(SEQ ID NO: 14)

| | |
|---|---|
| Motif VI: | [TI]XXXLYPNP[TK][GV]; |

(SEQ ID NO: 15)

| | |
|---|---|
| Motif VII: | E[HG]DXSX[ST]RXD; |
| | preferably, |
| Motif VII: | E[HG]DXSX[ST]RXD. |

In an embodiment, the aliphatic hydrocarbon which is substituted with a carboxy group is a fatty acid; preferably butanoic acid (butyric acid), pentanoic acid (valeric acid), hexanoic acid (caproic acid), heptanoic acid (enanthic acid), octanoic acid (caprylic acid), nonanoic acid (pelargonic acid), decanoic acid (capric acid), dodecanoic acid (lauric acid), tetradecanoic acid (myristic acid), hexadecanoic acid (palmitic acid), octadecanoic acid (stearic acid), eicosanoic acid (arachidic acid), linoleic acid, linolenic acid, arachidonic acid, eicosapentaenoic acid, or docosahexaenoic acid.

In an embodiment, the aliphatic hydrocarbon which is substituted with a carboxy group, is not lauric acid or palmitic acid.

In yet another aspect, the present invention also provides a method for changing (oxidizing) a primary alcohol of a linear or branched aliphatic hydrocarbon having at least five carbons to the corresponding acid, comprising contacting the alcohol of an aliphatic hydrocarbon with hydrogen peroxide and a polypeptide having peroxygenase activity; wherein the polypeptide comprises:

a) an amino acid sequence which has at least 30% identity to SEQ ID NO: 1, 2, 3, 4, 5, 6, 7, 8, 16, 17, 18, 19, 20, 21, 22, 23, 24, 25, 26, 27, 28 or 29; preferably SEQ ID NO:2 or SEQ ID NO:4; and
b) an amino acid sequence represented by one or more of the following motifs:

(SEQ ID NO: 9)

| | |
|---|---|
| Motif I: | [FL]XX[YF]S[AN]X[FHY]G[GN]GX[YF]N; |

(SEQ ID NO: 10)

| | |
|---|---|
| Motif II: | G[GN]GX[YF]NXX[VA]AX[EH][LF]R; |

(SEQ ID NO: 11)

| | |
|---|---|
| Motif III: | RXXRI[QE][DEQ]S[IM]ATN; |

(SEQ ID NO: 12)

| | |
|---|---|
| Motif IV: | S[IM]ATN[PG][EQN][FM][SDN][FL]; |

(SEQ ID NO: 13)

| | |
|---|---|
| Motif V: | P[PDK][DG]F[HFW]R[AP]; |

(SEQ ID NO: 14)

| | |
|---|---|
| Motif VI: | [TI]XXXLYPNP[TK][GV]; |

(SEQ ID NO: 15)

| | |
|---|---|
| Motif VII: | E[HG]DXSX[ST]RXD; |
| | preferably, |
| Motif VII: | E[HG]DXSX[ST]RXD. |

For example, pentanol may be changed (oxidized) to pentanoic acid (valeric acid), hexanol may be changed to hexanoic acid (caproic acid), heptanol may be changed to heptanoic acid (enanthic acid), octanol may be changed to octanoic acid (caprylic acid), nonanol may be changed to nonanoic acid (pelargonic acid), decanol may be changed to decanoic acid (capric acid), dodecanol may be changed to dodecanoic acid (lauric acid), tetradecanol may be changed to tetradecanoic acid (myristic acid), hexadecanol may be changed to hexadecanoic acid (palmitic acid), octadecanol may be changed to octadecanoic acid (stearic acid), and eicosanol may be changed to eicosanoic acid (arachidic acid).

The present invention is further described by the following examples that should not be construed as limiting the scope of the invention.

### EXAMPLES

The amino acid sequence of the peroxygenase from Agrocybe aegerita is shown as SEQ ID NO:2; and the amino acid sequence of the peroxygenase from Coprinopsis cinerea is shown as SEQ ID NO:4.

### Example 1

### Enzymatic Oxidation of Dodecane, Tetradecane and Hexadecane

The extracellular peroxygenase of A. aegerita (isoform II, 44 kDa, SEQ ID NO:2) was used. The enzyme preparation was homogeneous by sodium dodecylsulfate-polyacrylamide gel electrophoresis, an exhibited and A₄₁₈/A₂₈₀ ratio of 1.75. Its specific activity was 117 units·mg⁻¹, where 1 unit represents the oxidation of 1 μmol of veratryl alcohol to veratraldehyde (ε₃₁₀ 9300 M⁻¹·cm⁻¹) in 1 minute at 23° C. and pH 7, in the presence of 2.5 mM H₂O₂.

Three alkanes: dodecane (C₁₂), tetradecane (C₁₄) and hexadecane (C₁₆) were obtained from Sigma-Aldrich. Five mL reactions of the above model substrates (1 mM) with the A. aegerita peroxygenase (1 U) were performed in 50 mM sodium phosphate buffer (pH 7) at 25° C. for 2 h, in the presence of 2.5 mM H₂O₂. The substrates were previously dissolved in acetone and added to the buffer (the acetone concentration in the reaction was 15%). In control experiments, substrates were treated under the same conditions but without enzyme. After the enzymatic reactions, water was immediately removed in a rotary evaporator, and the products recovered with chloroform, dried under nitrogen, and redissolved in chloroform for GC-MS analyses. Bis(trimethylsilyl)trifluoroacetamide (Supelco) in the presence of pyridine was used to prepare trimethylsilyl derivatives.

The GC-MS analyses were performed with a Varian 3800 chromatograph coupled to an ion-trap detector (Varian 4000) using a medium-length fused-silica DB-5HT capillary column (12 m×0.25 mm internal diameter, 0.1 μm film thickness) from J&W Scientific, enabling simultaneous elution of the different compound classes. The oven was heated from 120° C. (1 minute) to 380° C. at 10° C. per minute, and held for 5 minutes. Other temperature program, from 50° C. to 110° C. (at 30° C. per minute) and then to 320° C. (at 6° C. per minute), was used when necessary. In all GC-MS analyses, the transfer line was kept at 300° C., the injector was programmed from 120° C. (0.1 minute) to 380° C. at 200° C.·per minute and held until the end of the analysis, and helium was used as carrier gas at a rate of 2 ml per minute.

Compounds were identified by mass fragmentography, and by comparing their mass spectra with those of the Wiley and NIST libraries and standards, and quantification was obtained from total-ion peak area, using response factors of the same or similar compounds. Single-ion chromatographic profiles (of base or other specific ions) were used to estimate compound abundances when two peaks partially overlapped.

### Results

Three saturated alkanes (dodecane, tetradecane and hexadecane) were tested as A. aegerita peroxygenase substrates.

TABLE 1
GC-MS peak areas for the peroxygenase reactions.

| | (ω-1) | (ω-2) | 2, (ω-1) | 2, (ω-2) | 3, (ω-2) | ω-1-OH- | ω-2-OH- |
|---|---|---|---|---|---|---|---|
| Substrate | OH | OH | di OH | di OH | di OH | (2 + 3 keto) | (2 + 3 keto) |
| dodecane | 90,000 | 18,000 | 2.4 × 10⁶ | 4.3 × 10⁶ | 2.1 × 10⁶ | 1.3 × 10⁶ | 1.1 × 10⁶ |
| tetradecane | 120,000 | 140,000 | 260,000 | 420,000 | 350,000 | 520,000 | 740,000 |
| hexadecane | 90,000 | 70,000 | 60,000 | 150,000 | 100,000 | 100,000 | 120,000 |

The reactions with dodecane, gave monohydroxylated derivatives at positions 2 and 3. In addition to the monohydroxylated derivatives, dihydroxylations at the positions 2 and 3 from both ends of the molecule (i.e., α+1 and ω-1-ω-2, or α+2 and ω-1-ω-2) were identified as the predominant compounds.

### Example 2

### Enzymatic Oxidation of Saturated and Unsaturated Fatty Acids

The extracellular peroxygenase of A. aegerita (isoform II, 44 kDa, SEQ ID NO:2) was used. The enzyme preparation was homogeneous by sodium dodecylsulfate-polyacrylamide gel electrophoresis, an exhibited and A₄₁₈/A₂₈₀ ratio of 1.75. Its specific activity was 117 units·mg⁻¹, where 1 unit represents the oxidation of 1 μmol of veratryl alcohol to veratraldehyde (ε₃₁₀ 9300 M⁻¹·cm⁻¹) in 1 minute at 23° C. and pH 7, in the presence of 2.5 mM H₂O₂.

Saturated and unsaturated acids were obtained from Sigma-Aldrich: Lauric (dodecanoic, C₁₂), myristic (tetradecanoic C₁₄), palmitic (hexadecanoic, C₁₆), stearic (octadecanoic, C₁₈), arachidic (eicosanoic, C₂₀), lauroleic (cis-9-dodecenoic, C_{12:1}), myristoleic (cis-9-tetradecenoic, C_{14:1}), palmitoleic (cis-9-hexadecenoic, C_{16:1}), oleic (cis-9-octadecenoic, C_{18:1}), linoleic (cis,cis-9,12-octadecadienoic, C_{18:2}) and eicosenoic (C_{20:1}) acids. Five mL reactions of the above model substrates (1 mM) with the A. aegerita peroxygenase (1 U) were performed in 50 mM sodium phosphate buffer (pH 7) at 25° C. for 2 hours, in the presence of 2.5 mM H₂O₂. The substrates were previously dissolved in acetone and added to the buffer (the acetone concentration in the reaction was 15%). In control experiments, substrates were treated under the same conditions but without enzyme. After the enzymatic reactions, water was immediately removed in a rotary evaporator, and the products recovered with chloroform, dried under nitrogen, and redissolved in chloroform for GC-MS analyses. Bis(trimethylsilyl)trifluoroacetamide (Supelco) in the presence of pyridine was used to prepare trimethylsilyl derivatives.

The GC-MS analyses were performed with a Varian 3800 chromatograph coupled to an ion-trap detector (Varian 4000) using a medium-length fused-silica DB-5HT capillary column (12 m×0.25 mm internal diameter, 0.1 μm film thickness) from J&W Scientific, enabling simultaneous elution of the different compound classes. The oven was heated from 120° C. (1 minute) to 380° C. at 10° C. per minute, and held for 5 minutes. Other temperature program, from 50° C. to 110° C. (at 30° C. per minute) and then to 320° C. (at 6° C. per minute), was used when necessary. In all GC-MS analyses, the transfer line was kept at 300° C., the injector was programmed from 120° C. (0.1 minute) to 380° C. at 200° C.·per minute and held until the end of the analysis, and helium was used as carrier gas at a rate of 2 ml per minute.

Compounds were identified by mass fragmentography, and by comparing their mass spectra with those of the Wiley and NIST libraries and standards, and quantification was obtained from total-ion peak area, using response factors of the same or similar compounds. Single-ion chromatographic profiles (of base or other specific ions) were used to estimate compound abundances when two peaks partially overlapped.

### Results

Eleven saturated and unsaturated fatty acids were tested as substrates of the A. aegerita peroxygenase and all fatty acids showed reactivity towards the enzyme. The abundance (relative percentage) of different monohydroxylated, keto, dihydroxylated, keto-hydroxy and dicarboxylic derivatives identified by GC-MS in the reactions of saturated and unsaturated fatty acids are listed in Table 2.

TABLE 2
Relative abundance of reaction products.

| Fatty | ω | ω-1 | ω-2 | ω-3 | ω-1 | ω-2 | | OH- | di- |
|---|---|---|---|---|---|---|---|---|---|
| acid | OH | OH | OH | OH | keto | keto | di-OH | keto | COOH |
| C₁₂ | 1.3 | 39.7 | 32.0 | 0.2 | 5.8 | 1.0 | 4.4 | 15.5 | 0.3 |
| C_{12:1} | 3.3 | 37.4 | 59.2 | 0 | <0.1 | <0.1 | 0 | 0 | 0 |
| C₁₄ | 3.5 | 34.4 | 30.5 | 0.3 | 20.8 | 3.3 | 0.5 | 6.2 | 0.6 |
| C_{14:1} | 1.8 | 0 | 94.6 | 0 | 0 | 3.6 | 0 | 0 | 0 |
| C₁₆ | 1.4 | 23.6 | 23.6 | 0.3 | 34.5 | 16.3 | 0 | 0 | 0.3 |
| C_{16:1} | 2.5 | 35.7 | 47.0 | 0.1 | 10.4 | 4.4 | 0 | 0 | 0 |
| C₁₈ | <0.1 | 22.7 | 27.0 | 0.1 | 32.8 | 17.0 | 0 | 0 | 0.5 |
| C_{18:1} | 1.6 | 40.8 | 39.0 | 0.2 | 13.0 | 5.3 | 0 | 0 | 0 |
| C_{18:2} | 1.0 | 50.2 | 33.5 | 2.5 | 10.0 | 2.9 | 0 | 0 | 0 |
| C₂₀ | <0.1 | 16.0 | 28.1 | 0 | 38.7 | 17.3 | 0 | 0 | 0 |
| C_{20:1} | 1.2 | 35.0 | 38.7 | 0.4 | 18.8 | 6.0 | 0 | 0 | 0 |

Oxidation of the terminal methyl group (w OH) was observed for all tested free fatty acids, in some cases this was further oxidized leading to formation of dicarboxylic acids (di-COOH).

### Example 3

### Enzymatic Oxidation of Tetradecane in 40% Acetone

The extracellular peroxygenase of A. aegerita (isoform II, 44 kDa, SEQ ID NO:2) and the recombinant peroxygenase of Coprinopsis cinerea (WT392, SEQ ID NO:4) were used. The activity of the preparations was determined by oxidation of veratryl alcohol. 1 unit represents the oxidation of 1 μmol of veratryl alcohol to veratraldehyde (ε₃₁₀ 9300 M⁻¹·cm⁻¹) in 1 minute at 23° C. and pH 7, in the presence of 2.5 mM H₂O₂.

Tetradecane (C₁₄) was obtained from Sigma-Aldrich. Five mL reactions of the above model substrate (0.3 mM) with 1 U of peroxygenase were performed in 50 mM sodium phosphate buffer (pH 7) at 40° C. for 2 h, in the presence of H₂O₂. The concentration of H₂O₂ was 2.5 mM when A. aegerita peroxygenase was applied and 0.5 mM when using C. cinerea peroxygenase. The substrate was previously dissolved in acetone and added to the buffer (the acetone concentration in the reaction was 40%). In control experiments, substrates were treated under the same conditions but without enzyme. After the enzymatic reactions, water was immediately removed in a rotary evaporator, and the products recovered with chloroform, dried under nitrogen, and redissolved in chloroform for GC-MS analyses. Bis(trimethylsilyl)trifluoroacetamide (Supelco) in the presence of pyridine was used to prepare trimethylsilyl derivatives.

The GC-MS analyses were performed with a Varian 3800 chromatograph coupled to an ion-trap detector (Varian 4000) using a medium-length fused-silica DB-5HT capillary column (12 m×0.25 mm internal diameter, 0.1 μm film thickness) from J&W Scientific, enabling simultaneous elution of the different compound classes. The oven was heated from 120° C. (1 minute) to 380° C. at 10° C. per minute, and held for 5 minutes. Other temperature program, from 50° C. to 110° C. (at 30° C. per minute) and then to 320° C. (at 6° C. per minute), was used when necessary. In all GC-MS analyses, the transfer line was kept at 300° C., the injector was programmed from 120° C. (0.1 minute) to 380° C. at 200° C.·per minute and held until the end of the analysis, and helium was used as carrier gas at a rate of 2 ml per minute.

Compounds were identified by mass fragmentography, and by comparing their mass spectra with those of the Wiley and NIST libraries and standards, and quantification was obtained from total-ion peak area, using response factors of the same or similar compounds. Single-ion chromatographic profiles (of base or other specific ions) were used to estimate compound abundances when two peaks partially overlapped.

### Results

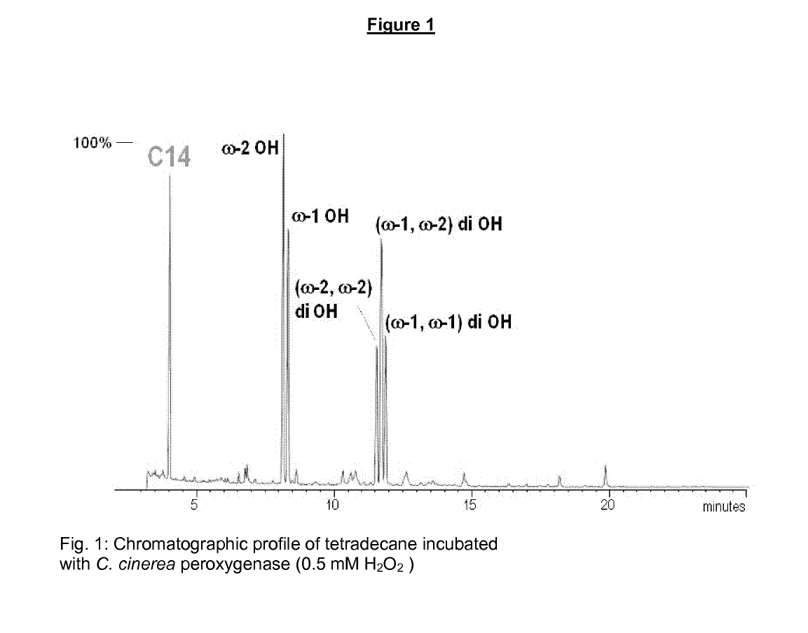

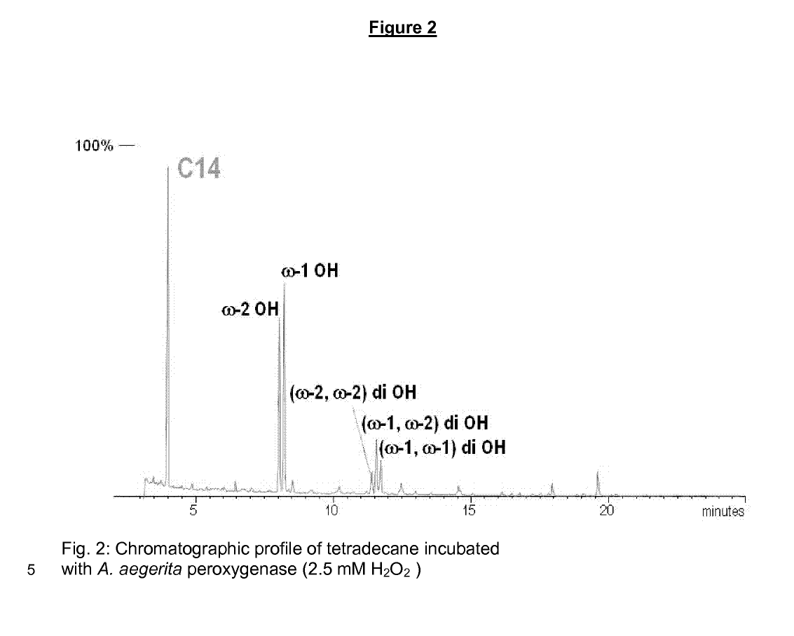

Chromatographic profiles resulting from hydroxylation of the saturated alkane tetradecane (C14) are shown in FIG. 1 for C. cinerea peroxygenase and FIG. 2 for A. aegerita peroxygenase.

The reactions with tetradecane, resulted in monohydroxylated derivatives at positions 2 and 3 (ω-1 and ω-20H) and dihydroxylations at the positions 2 and 3 from both ends of the molecule (i.e., ω-1/ω-1, ω-2/ω-2 or ω-1/ω-2 di OH).

### Example 4

### Enzymatic Oxidation of 1-Tetradecanol in 20% Acetone

The extracellular peroxygenase of A. aegerita (isoform II, 44 kDa, SEQ ID NO: 2) and recombinant peroxygenase of Coprinopsis cinerea (WT392, SEQ ID NO: 4) were used. The activity of the preparations was determined by oxidation of veratryl alcohol. 1 unit represents the oxidation of 1 μmol of veratryl alcohol to veratraldehyde (ε₃₁₀ 9300 M⁻¹·cm⁻¹) in 1 minute at 23° C. and pH 7, in the presence of 2.5 mM H₂O₂.

1-Tetradecanol (C₁₄) was obtained from Sigma-Aldrich. Five mL reactions of the above model substrate (0.1 mM) with 1 U of peroxygenase were performed in 50 mM sodium phosphate buffer (pH 7) at 30° C. for 1 minute, in the presence of H₂O₂. The concentration of H₂O₂ was 2.5 mM when A. aegerita peroxygenase was applied and 0.5 mM when using C. cinerea peroxygenase. The substrate was previously dissolved in acetone and added to the buffer (the acetone concentration in the reaction was 20%). In control experiments, substrates were treated under the same conditions but without enzyme. After the enzymatic reactions, water was immediately removed in a rotary evaporator, and the products recovered with chloroform, dried under nitrogen, and redissolved in chloroform for GC-MS analyses. Bis(trimethylsilyl)trifluoroacetamide (Supelco) in the presence of pyridine was used to prepare trimethylsilyl derivatives.

The GC-MS analyses were performed with a Varian 3800 chromatograph coupled to an ion-trap detector (Varian 4000) using a medium-length fused-silica DB-5HT capillary column (12 m×0.25 mm internal diameter, 0.1 μm film thickness) from J&W Scientific, enabling simultaneous elution of the different compound classes. The oven was heated from 120° C. (1 minute) to 380° C. at 10° C. per minute, and held for 5 minutes. Other temperature program, from 50° C. to 110° C. (at 30° C. per minute) and then to 320° C. (at 6° C. per minute), was used when necessary. In all GC-MS analyses, the transfer line was kept at 300° C., the injector was programmed from 120° C. (0.1 minute) to 380° C. at 200° C.·per minute and held until the end of the analysis, and helium was used as carrier gas at a rate of 2 ml per minute.

Compounds were identified by mass fragmentography, and by comparing their mass spectra with those of the Wiley and NIST libraries and standards, and quantification was obtained from total-ion peak area, using response factors of the same or similar compounds. Single-ion chromatographic profiles (of base or other specific ions) were used to estimate compound abundances when two peaks partially overlapped.

### Results

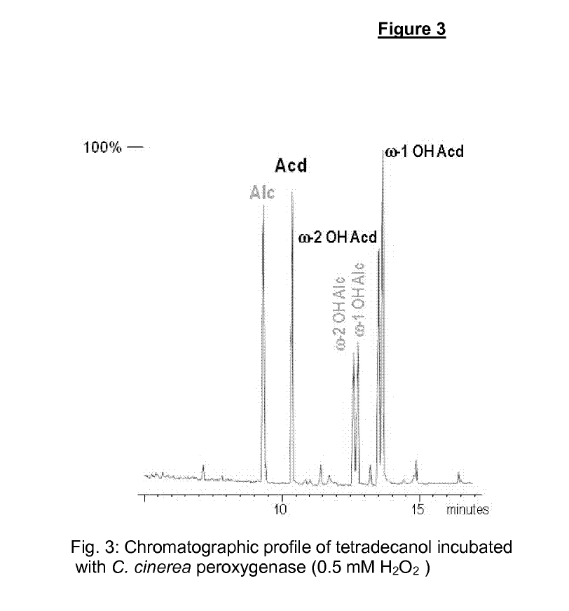

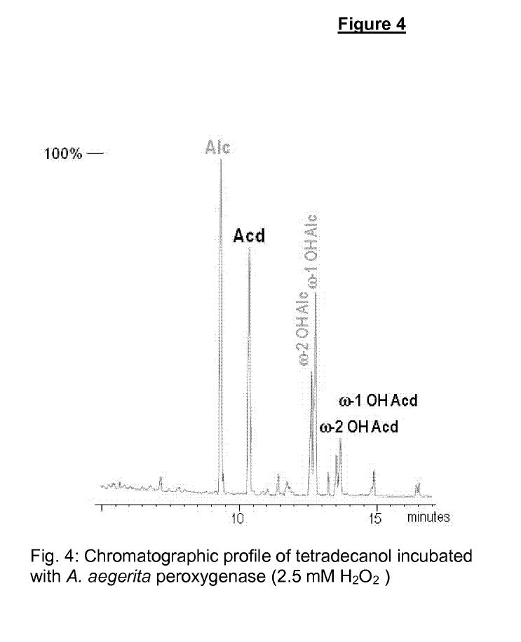

Chromatographic profiles resulting from oxidation of 1-tetradecanol (Alc) are shown in FIG. 3 for C. cinerea peroxygenase and FIG. 4 for A. aegerita peroxygenase.

The reactions with 1-tetradecanol, resulted in formation of 1-tetradecanoic acid (Acd) and hydroxylated decanoic acid (ω-10H Acd and ω-20H Acd) and two dihydroxylated products (ω-1 OH Alc and ω-2 OH Alc).

1. A method for introducing a hydroxy or a keto group at the second or third carbon of at least two ends of a substituted or unsubstituted, linear or branched, aliphatic hydrocarbon having at least five carbons and having at least one hydrogen attached to said second or third carbon, comprising contacting the aliphatic hydrocarbon with hydrogen peroxide and a polypeptide having peroxygenase activity; wherein the polypeptide comprises:
a) an amino acid sequence which has at least 30% identity to SEQ ID NO: 1, 2, 3, 4, 5, 6, 7, 8, 16, 17, 18, 19, 20, 21, 22, 23, 24, 25, 26, 27, 28 or 29; and
b) an amino acid sequence represented by one or more of the following motifs:
(SEQ ID NO: 9)

| | |
|---|---|
| Motif I: | [FL]XX[YF]S[AN]X[FHY]G[GN]GX[YF]N; |

(SEQ ID NO: 10)

| | |
|---|---|
| Motif II: | G[GN]GX[YF]NXX[VA]AX[EH][LF]R; |

(SEQ ID NO: 11)

| | |
|---|---|
| Motif III: | RXXRI[QE][DEQ]S[IM]ATN; |

(SEQ ID NO: 12)

| | |
|---|---|
| Motif IV: | S[IM]ATN[PG][EQN][FM][SDN][FL]; |

(SEQ ID NO: 13)

| | |
|---|---|
| Motif V: | P[PDK][DG]F[HFW]R[AP]; |

(SEQ ID NO: 14)

| | |
|---|---|
| Motif VI: | [TI]XXXLYPNP[TK][GV]; |

(SEQ ID NO: 15)

| | |
|---|---|
| Motif VII: | E[HG]DXSX[ST]RXD. |

2. The method of claim 1, wherein the second or third carbon is unsubstituted until contacted with the peroxygenase.

3. The method of claim 1, wherein the polypeptide comprises or consists of an amino acid sequence having to the mature polypeptide of SEQ ID NO: 1, 2, 3, 4, 5, 6, 7, 8, 16, 17, 18, 19, 20, 21, 22, 23, 24, 25, 26, 27, 28 or 29.

4. The method of claim 1, wherein the polypeptide comprises or consists of the amino acid sequence of SEQ ID NO: 1, 2, 3, 4, 5, 6, 7, 8, 16, 17, 18, 19, 20, 21, 22, 23, 24, 25, 26, 27, 28 or 29.

5. The method of claim 1, wherein the substituents of the aliphatic hydrocarbon are selected from the group consisting of halogen, hydroxyl, carboxyl, amino, nitro, cyano, thiol, sulphonyl, formyl, acetyl, methoxy, ethoxy, phenyl, benzyl, xylyl, carbamoyl and sulfamoyl.

6. The method of claim 1, wherein the substituents are selected from the group consisting of chloro, hydroxyl, carboxyl and sulphonyl; in particular chloro and carboxyl.

7. The method of claim 1, wherein the aliphatic hydrocarbon is an alkane.

8. The method of claim 7, wherein the alkane is pentane, hexane, heptane, octane, nonane, decane, undecane, dodecane, tridecane, tetradecane, pentadecane or hexadecane, or isomers thereof.

9. The method of claim 7, wherein the alkane is undecane, dodecane, tridecane, tetradecane, pentadecane or hexadecane, or isomers thereof.

10. The method of claim 1, wherein the aliphatic hydrocarbon is unsubstituted.

11. The method of claim 1, wherein the aliphatic hydrocarbon is linear.

12. The method of claim 1, wherein the aliphatic hydrocarbon is converted to a diol, by introduction of two hydroxy groups.

13. An enzymatic method for producing polyurethane, comprising converting an aliphatic hydrocarbon to a diol, according to the method of claim 12, and using the diol for producing polyurethane.

14-15. (canceled)

16. The method of claim 1, wherein the polypeptide comprises or consists of an amino acid sequence having at least 80% identity to the mature polypeptide of SEQ ID NO: 1, 2, 3, 4, 5, 6, 7, 8, 16, 17, 18, 19, 20, 21, 22, 23, 24, 25, 26, 27, 28 or 29.

17. The method of claim 1, wherein the polypeptide comprises or consists of an amino acid sequence having at least 90% identity to the mature polypeptide of SEQ ID NO: 1, 2, 3, 4, 5, 6, 7, 8, 16, 17, 18, 19, 20, 21, 22, 23, 24, 25, 26, 27, 28 or 29.

18. The method of claim 1, wherein the polypeptide comprises or consists of an amino acid sequence having at least 95% identity to the mature polypeptide of SEQ ID NO: 1, 2, 3, 4, 5, 6, 7, 8, 16, 17, 18, 19, 20, 21, 22, 23, 24, 25, 26, 27, 28 or 29.

19. The method of claim 1, wherein the polypeptide comprises or consists of the amino acid sequence of SEQ ID NO: 2.

20. The method of claim 1, wherein the polypeptide comprises or consists of the amino acid sequence of SEQ ID NO: 4.
